(19) United States
(12) Patent Application Publication
(10) Pub. No.: US 2025/0128014 A1
(43) Pub. Date: Apr. 24, 2025

(54) AUTOMATED VENTILATOR SYSTEM WITH DUAL-VALVE PEEP ASSEMBLY AND SOFTWARE WATCHDOG

(71) Applicant: BurLeon Tech LLC, Houston, TX (US)

(72) Inventors: Atuhani Seth BURNETT, Whitefish Bay, WI (US); Rod Diagan GONZAGA, Scarborough (CA); Christopher Mikal LOGAN, Goshen, NY (US); Jehon Sherman LEONCE, Houston, TX (US); Faraz Shaukat ALI, Houston, TX (US)

(21) Appl. No.: 18/381,927

(22) Filed: Oct. 19, 2023

(51) Int. Cl. A61M 16/20; A61M 16/00; G16H 40/63

(52) U.S. Cl. CPC A61M 16/205; G16H 40/63; A61M 2016/0027; A61M 2016/0033; A61M 2205/502; A61M 2230/06; A61M 2230/432

(57) ABSTRACT

A control unit for controlling a ventilator system includes a memory and a control system. The memory contains machine readable medium comprising machine executable code having stored thereon instructions. The control system is coupled to the memory and includes processor(s). The control system is configured to execute the machine executable code to cause the control system to identify one or more hardware components connected to the ventilator system; select one or more corresponding drivers to run the one or more identified hardware; receive, via a user interface, a ventilator control mode; create an instance of the ventilator control mode; create, based on the ventilator control mode, an instance for each aforementioned cycles, wherein each instance includes instructions for at least one hardware component; and signal the instance for each of the inhale cycle, the exhale cycle, and the wait cycle to perform function in loop.

### FIELD OF THE INVENTION

The present invention relates generally to an automated ventilator system, and more specifically, to a software-controlled ventilator system with variable control modes.

### BACKGROUND OF THE INVENTION

A ventilator is a device that can replace, control, or alter a person's normal physiological breathing and is often used to increase the patient's lung ventilation, improve the patient's respiratory function, reduce respiratory power consumption, and conserve the patient's cardiac reserve capacity. As such, the ventilators are safety-critical devices that help patients breathe that are often used in hospital intensive care units (ICUs), operating rooms, transport between hospital units, and pre-hospital transport.

Ventilators work by delivering oxygen directly to the lungs and simultaneously pump out carbon dioxide for patients who are unable to inhale or exhale on their own. The ventilator delivers oxygen via a tube that is inserted through the patient's nose or mouth in a procedure known as intubation or that is placed directly into the trachea, or windpipe, in a surgical procedure known as tracheostomy. The opposite end of the tube is connected to the ventilator machine that pumps a mixture of medical air and oxygen through the tube and into the lungs. The medical air is often warmed and humidified before it goes into the patient's body.

The ventilator further plays a vital role in maintaining positive airway pressure to help prevent small air sacs (i.e., alveoli) in the lungs from collapsing. During unassisted exhalation, a patient's alveoli contract but do not completely collapse, partly due to the presence of a substance called surfactant, which is secreted by the lungs and is an agent that decreases the surface tension between two media. This decreases the surface tension within the alveoli to ensure that complete collapse does not take place. When a patient is placed on ventilator, however, ventilation of the patient tends to inactivate the pulmonary surfactant which then leads to collapse of some or all of the alveoli. If these collapsed alveoli are not re-expanded during the next inhalation cycle, they remain unavailable for gas exchange, making oxygenation of the patient more difficult as the surface area of the lung is now reduced. Thus, most ventilator systems use various mechanisms to apply a Positive End-Expiratory Pressure (“PEEP”) to the patient's lungs at the end of each exhalation—that is, a minimum amount of pressure is provided within the patient's lungs to prevent collapse of the alveoli, and thus maintain the gas exchange capacity of the lungs. To control pressure in the patient's lungs during exhalation, the rate that air is exhaled is closely regulated to ensure that some air remains within the patient's lungs at the end of an exhalation. Various types of PEEP exhale valves have been designed to achieve this purpose. Many of these types of valves use mechanical springs to balance the pressure in the lungs with atmospheric pressure. Some use electronic valves which attempt to close at exactly the right instant to catch and maintain the down trending lung pressure at the desired peep pressure. If the valve opens widely, the pressure may drop too fast and therefore catching the pressure at the right moment may be unreliable. On the other hand, if the valve opens too narrowly and the pressure drops slowly, the ventilator may take too long to completely empty the patient's lungs. One solution could be to use a variable aperture valve, however, the apertures for these valves are typically small and require high pressure for appreciable gas flow.

A second issue is that ventilators are safety-critical devices. Ideally, the ventilator system would be able to run all its components simultaneously to ensure that readings taken by various sensors are immediately taken into account and reflected in the settings of the ventilator. Further, on a related note, it is imperative that the ventilator is fail-proof and able to detect any internal failures or irregularities in a timely manner, and notify the user, to avoid harming the patient.

As third issue, the emergence of COVID-19 as a common infectious respiratory disease has placed a significant strain on the availability of trained personnel to operate ventilators, doing tasks such as entering appropriate ventilator settings based on the patient's condition. In most scenarios where ventilators are used, an ICU or anesthesia physician makes the initial determination of the ventilator settings. Subsequently, based on the pH and dissolved CO2/O2 content of the blood gas analysis, ongoing adjustments are made to the ventilator oxygen mix (FiO2), PEEP setting, delivered volume setting (tidal volume), respiratory rate, and possibly the amount of pressure support the patient is receiving if they are breathing spontaneously. This analysis is critical to ongoing ventilator support, but without a physician present to make such adjustments, it is challenging to properly implement the appropriate settings as the patient's lung conditions change. Thus, it is also imperative to develop a ventilator with the ability to provide initial recommended ventilator settings and subsequently make ongoing recommendations to adjust settings based on patient data.

Additionally, to improve versatility of the ventilator, a ventilator needs to be able to operate under different ventilation modes depending on the patient's needs.

### SUMMARY OF THE INVENTION

In view of the foregoing problems, the present disclosure describes an automated ventilator system with several key innovations that resolve the above problems. First, the present disclosure describes an automated ventilator system with a modular gas pathway and valve system that includes (1) a gas supply module, (2) a gas intake control and mixing module, (3) a patient delivery module, and (4) an exhalation module. The exhalation module includes a novel dual-valve PEEP assembly configured to regulate the air pressure in and the rate that air is exhaled from the patient's lungs. This is achieved through the use of a valve assembly that includes a rapid (flow rate) valve and a slow (flow rate) valve connected to a microprocessor that receives data on how much gas is left in the patient's lungs. Second, the present disclosure describes an overlapping three schema software architecture that allows multiple ventilator functions to run simultaneously as multiple, independent computational threads. Using this configuration, each ventilator function runs independently and does not interfere with the operation of other ventilator functions, thus allowing the various ventilator sensors to detect and the ventilator control unit to adjust settings based on a constant stream of uninterrupted, near-instantaneous sensor data. Thirdly, the present disclosure further discloses a software watchdog function that monitors for software failures by detecting a continuous or periodic heartbeat signal indicative of normal operation with one or more computational threads that run the ventilator components. If the software watchdog does not receive a heartbeat signal within a predefined timer interval, the software watchdog is configured to automatically terminate and restart the ventilator software with the last registered parameters and continue to operate the ventilator with minimal interruption. The present disclosure also discloses smart assistant functionality which allows the ventilator system to recommend initial settings for the ventilator and make ongoing suggestions/changes to the ventilator settings based on the blood gas values of the patient, thus substituting some of the need to have specially trained personnel to determine the initial ventilator settings and make subsequent adjustments.

Throughout this application, the term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to certain aspects of the present disclosure, a control unit for controlling a ventilator system includes a memory and a control system. The memory contains machine readable medium comprising machine executable code having stored thereon instructions. The control system is coupled to the memory and includes one or more processors. The control system is configured to execute the machine executable code to cause the control system to identify one or more hardware components connected to the ventilator system. The control system is also configured to select one or more corresponding drivers to run the one or more identified hardware. The control system is further configured to receive, via a user interface, a ventilator control mode. The control system is further configured to create an instance of the ventilator control mode. The control system is further configured to create, based on the ventilator control mode, an instance for each of an inhale cycle, an exhale cycle, and a wait cycle, wherein each instance includes instructions for at least one hardware component of the one or more hardware components. The control system is further configured to signal the instance for each of the inhale cycle, the exhale cycle, and the wait cycle to perform function in loop.

According to another aspect of the control unit described above, the control system is further configured to receive, via the user interface, a command to stop the ventilator control mode. The control system is then further configured to terminate each of the inhale cycle, the exhale cycle, and the wait cycle, in turn.

According to another aspect of the control unit described above, the one or more hardware components include at least one of a flow meter, a pressure sensor, a carbon dioxide sensor, a variable aperture gas valve, a binary open/close gas valve.

According to another aspect of the control unit described above, the functions are compartmentalized into a plurality of independent computational threads.

According to another aspect of the control unit described above, the plurality of independent computation threads include at least one of a ventilator master controller, a ventilator control mode, a scheduler for an inter-integrated circuit bus, an error detection watchdog, a graphical user interface, and a signal stimulator for the ventilator software. In some embodiments, independent computational threads for the inhale, exhale, and wait cycles of the ventilator are instantiated and de-constructed as needed to complete their specific tasks. According to some embodiments, the duration of the computational threads for inhale, exhale, and wait cycles are short-lived-being instantiated and de-constructed within the same ventilation cycle.

According to another aspect of the control unit described above, the ventilator control mode includes at least one of a volume control mode, a continuous positive airway pressure (CPAP) mode, synchronized intermittent mandatory ventilation (SIMV) mode, and a standby mode.

According to another aspect of the control unit described above, the control unit further includes a heartbeat generator unit configured to generate a periodic electronic signal based on receiving register signal from one or more independent computational threads.

According to another aspect of the control unit described above, the control unit further includes a heartbeat monitor unit configured to terminate and restart a main ventilator thread with the last registered parameters if the periodic electronic signal is not received within a predefined time interval.

According to certain aspects of the present disclosure, a positive end-expiratory pressure (PEEP) exhale valve assembly includes a tubing, a pressure sensor, a flow meter, a valve assembly, and a control system. The tubing has a first end adapted to receive exhaled air from a patient and a second end adapted to expel the exhaled air. The pressure sensor and the flow meter are configured to detect air pressure within the tubing and flow rate of the exhaled air through the tubing, respectively. The valve assembly encapsulates the second end of the tubing. The valve assembly includes a first valve that releases the exhaled air at a first rate when opened, and a second valve that releases the exhaled air at a second rate when opened. The second rate being lower than the first rate. The control system configured to toggle the first valve when a plurality of parameters reach a first trigger threshold and toggle the second valve when the plurality of parameters reach a second trigger threshold, the plurality of parameters being based on the air pressure detected by the pressure sensor, the flow rate detected by the flow meter, and the volume of air that has been exhaled.

According to another aspect of the PEEP exhale assembly described above, the valve assembly further includes a manifold having an input terminal connected to the second end of the tubing, a first output terminal connected to the first valve and a second output terminal connected to the second valve.

According to another aspect of the PEEP exhale assembly described above, the first valve has a first aperture and the second valve has a second aperture, wherein the second aperture is smaller than the first aperture.

According to another aspect of the PEEP exhale assembly described above, the aperture of the second valve is configured to release the exhaled air at a rate for maintaining the user defined positive end-expiratory pressure in lungs of the patient.

According to another aspect of the PEEP exhale assembly described above, the first valve and the second valve are solenoid type valves. According to some embodiments the solenoid type valves are binary open/close valves.

According to another aspect of the PEEP exhale assembly described above, the assembly further includes a carbon dioxide monitor configured to detect a carbon dioxide level in the exhaled air.

According to another aspect of the PEEP exhale assembly described above, the first valve and the second valve are open at the initiation of exhalation by the patient.

According to another aspect of the PEEP exhale assembly described above, the first valve closes when, based on measurements of the flow meter, a volume of exhaled air reaches a predetermined proportion of a volume of previously inhaled air. The control unit of a ventilator system uses the measurements taken by the flow meter to keep track of the volume of exhaled air breathed out by a patient. Then, the control unit compares the volume of exhaled air to a volume of previously inhaled air. The first valve closes when the volume of exhaled air reaches a predetermined proportion of the volume of previously inhaled air.

According to another aspect of the PEEP exhale assembly described above, the second valve closes when a pressure in the tubing decrease to a predetermined positive end-expiratory pressure.

According to another aspect of the PEEP exhale assembly described above, the pressure in the tubing is indicated by pressure data from the pressure sensor. According to some embodiments, the control unit compares the continuous pressure data taken from the pressure sensor to the predetermined PEEP pressure. The control unit closes the second valve when the pressure data taken by the pressure sensor indicates that the pressure within the tubing has dropped to the predetermined pressure.

According to other aspects of the present disclosure, a method of controlling exhalation of a patient by mechanical ventilator includes providing a positive end-expiratory pressure (PEEP) exhale valve assembly. The assembly includes a tubing, a pressure sensor, a flow meter, and a valve assembly. The tubing has a first end adapted to receive exhaled air from a patient and a second end adapted to expel the exhaled air. The pressure sensor and the flow meter are configured to detect air pressure within the tubing and flow rate of the exhaled air through the tubing, respectively. The valve assembly encapsulates the second end of the tubing. The valve assembly includes a first valve that releases the exhaled air at a first rate when opened, and a second valve that releases the exhaled air at a second rate when opened. The second rate is lower than the first rate. The method further includes opening, via the control system, the first valve and the second valve when a trigger event is registered by the control system. According to some embodiments, the trigger event is the initiation of an exhale cycle. According to some embodiments, the initiation of an exhale cycle is when the patient, or the system, initiates exhalation. The method further includes detecting, via the pressure sensor, the air pressure within the tubing. The method further includes detecting, via the flow meter, the flow rate of the exhaled air through the tubing, and by calculation, the volume of air being exhaled through the tubing. The method further includes closing, via a control system, the first valve when a volume of exhaled air reaches a predetermined proportion of a volume of previously inhaled air. According to some embodiments, the control system calculates the volume of exhaled air based on one or more measurements taken by the flow meter. The control system compares the volume of exhaled air to the volume of previously inhaled air. The control system closes the first valve when the volume of exhaled air reaches the predetermined proportion of the volume of inhaled air.

According to another aspect of the method described above, the method further includes closing, via the control system, the second valve when the pressure in the tubing, as measured by the pressure sensor, reaches a predetermined PEEP pressure. According to some embodiments, the predetermined PEEP pressure is a user-defined PEEP pressure.

### BRIEF DESCRIPTION OF THE DRAWINGS

The present disclosure, and its advantages, will be better understood from the following description of representative embodiments together with reference to the accompanying drawings. These drawings depict only representative embodiments and are therefore not to be considered as limitations on the scope of the various embodiments or claims.

FIG. 1 illustrates a functional block diagram of a ventilator system, according to some embodiments of the present disclosure.

FIG. 2 illustrates a schematic of an exemplary gas pathway for the ventilator system of FIG. 1, according to certain aspects of the present disclosure.

FIG. 3 illustrates an exemplary control software structure for controlling the ventilator system of FIG. 1, according to certain aspects of the present disclosure.

FIG. 4 illustrates exemplary computational threads for the ventilator system of FIG. 1 executing in parallel with each other, according to certain aspects of the present disclosure.

FIG. 5 illustrates an exemplary block diagram of a process for the ventilator system of FIG. 1 to suggest initial ventilator settings, according to some embodiments of the present disclosure.

FIG. 6 illustrates a block diagram of an exemplary software watchdog functionality for the ventilator system of FIG. 1, according to some embodiments of the present disclosure.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

For the present disclosure, the terms “computer system” or “computer device” or “computing system” refer to any electronically powered or battery-powered equipment that has hardware, software, and/or firmware components, where the software and/or firmware components can be configured for operating features on the device.

### System Overview

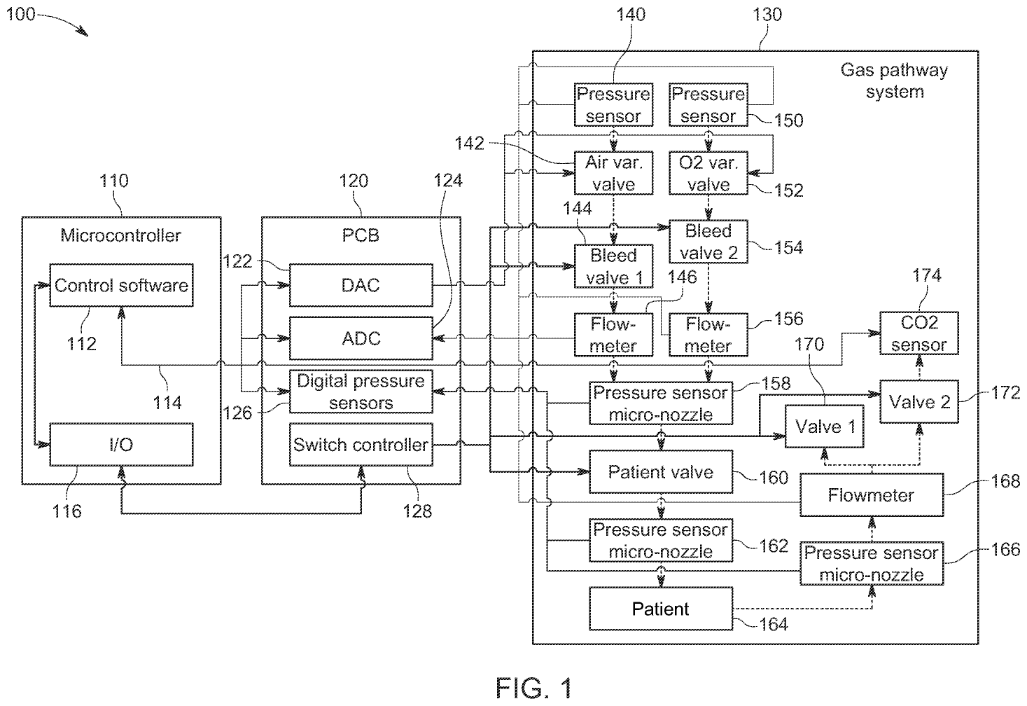

FIG. 1 illustrates a functional block diagram of an exemplary ventilator system 100, according to some embodiments of the present disclosure. According to some embodiments, the ventilator system 100 includes a gas pathway system 130 positioned between a patient and a gas source, a control unit 110 (e.g., a microcontroller) for controlling the gas pathway system 130, and a control board 120 onto which the control unit 110 is installed. For example, according to some embodiments, the control unit 110 can include one or more Raspberry Pi microcontrollers coupled to the control board 120 allowing one or more input-output (I/O) pins 116 direct access to the control board 120, and for the control board 120 to directly power the control unit 110. The control unit 110 further has installed thereon a control software 112 for exerting direct control over the control board 120 and, by extension, the gas pathway system 130. The control unit 110 is further connected to an inter-integrated circuit (I2C) bus 114 configured to relay data between the control unit 110 and one or more ventilator components. According to some embodiments, the I2C bus 114 originates from, and is controlled by the control unit 110. According to some embodiments, the I2C bus 114 also includes any wires or conductors connected directly or connected via other conductors to the I2C bus 114 pins on the control unit 110. Ventilator components in electrical communication with the control unit 110 via the I2C bus 114 can include, but is not limited to, components such as one or more digital pressure sensors 126, the multi-channel analog to digital converter (ADC) 124, one or more digital to analog converters (DAC) 122, a CO2 sensor 174, and flow meters 146,156, and 168 when they are operating in digital mode. Further, according to some implementations, each component connected to the I2C bus 114 has a unique address, allowing each component to be controlled independently by the control unit 110. The unique address of each component is defined in a ventilator setup file.

According to some embodiments, the control board 120 includes one or more digital to analog converters (DAC) 122, one or more analog to digital converters (ADC) 124, and one or more digital pressure sensors 126. Each of the foregoing components on the control board 120 are in electrical communication with the control unit 110 via the I2C bus 114. According to some embodiments, the one or more digital pressure sensors 126 include a series of digital pressure sensor chips residing directly on the control board and are connected directly to the I2C bus 114. Each pressure sensor chip has one or more pressure sensing micro-nozzles residing on the corresponding pressure sensor chip, which are connected to the gas pathway via one or more small caliber tubing, which are in turn connected to micro-nozzles from the appropriate portion of the gas pathway 158, 162, and 166.

According to some embodiments, the one or more analog to digital converters (ADC) 124 can include a multi-channel ADC chip configured to measure voltage signals on one or more analog sensors. For example, this can be implemented by using an 8-channel ADC to measure voltages ranging from 0 to 5 VDC on its channels, with at least some of the channels connected to analog sensors. The analog sensors can include components such as flow meters 146, 156 and 168 when they are operating in analog mode, as well as analog pressure sensors 140 and 150 used for measuring air and oxygen head pressures. The ADC chip resides directly on the control board 120 and is connected to the control unit 110 via the I2C bus 114. The control software 112 can request digital reading of the voltage on any of the channels to determine a measurement from the connected analog sensors.

According to some embodiments, the one or more digital to analog converters (DAC) 122 can include two DAC chips residing on the control board 120 and connected to the control unit 110 via the I2C bus 114. The DAC chips can be used to generate high-precision signal voltages that control the aperture of variable valves 142 and 152, which regulate the flow of medical air and oxygen, respectively.

According to some embodiments, the one or more switch controls 128 can be connected to the control unit 110 via the input/output (I/O) pins 116. The one or more switch controls 128 are further connected to one or more valves to regulate their opening. For example, the one or more switch controls can be connected to the bleed valves 144 and 154, the patient valve 160, and PEEP exhale valves 170 and 172 (i.e., a rapid valve and a slow valve). An exemplary implementation of the switching control can include a series of electronic relays that step up a 3 VDC from the I/O pins 116 of the control unit 110 to 12 VDC that regulate the on-and-off of various components of the ventilator system, such as the bleed valves 144 and 154, the patient valve 160, and the PEEP exhale valves 170 and 172. Additionally, the series of electronic relays can include a relay that controls power to the various chips residing on the control board 120 (e.g., the ADC chip 124, the DAC chips 122, and the digital pressure sensor chips 126), allowing the control unit 110 to toggle on-and-off each chip synchronously or independently.

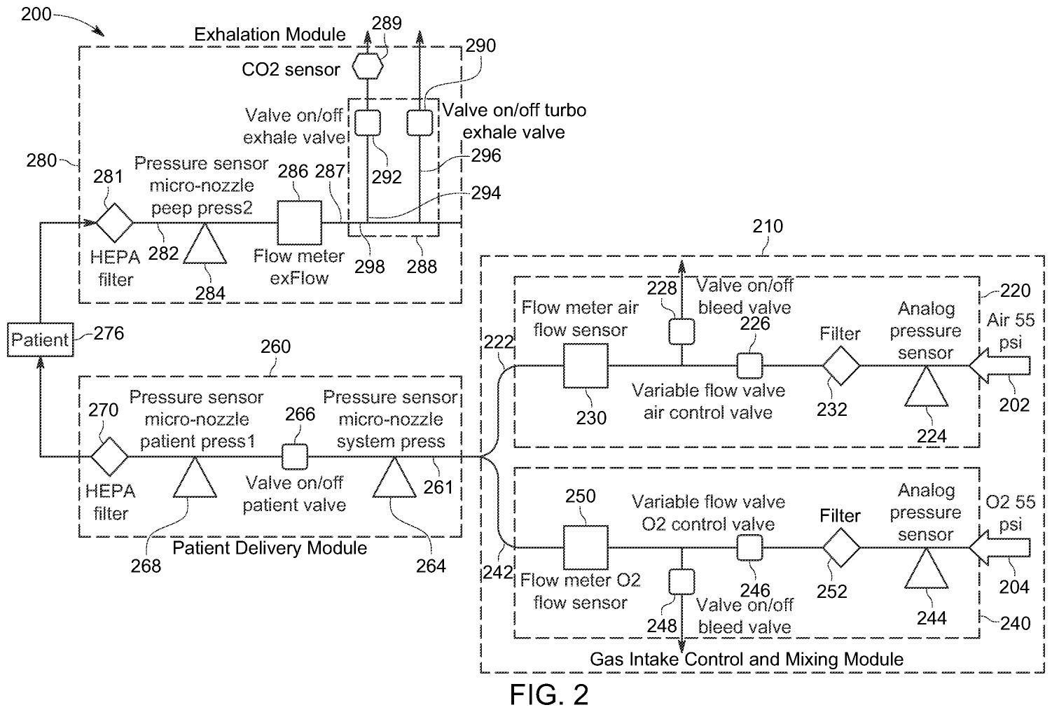

The gas pathway system 130 and the interaction between its components are shown in FIG. 2 and described in detail below.

### Ventilator Gas Pathway System

FIG. 2 illustrates a modular gas pathway system 200 for the ventilator system disclosed in the present application. The gas pathway system 200 feeds oxygenated air to the patient via a HEPA filter 270. The modular gas pathway system 200 includes a gas intake control and mix delivery module 210, a patient delivery module 260, and an exhalation module 280. According to some embodiments, each of the patient delivery module 260 and the exhalation module 280 includes a HEPA filter (270 and 281) adapted for connecting to a patient's mask or endotracheal tube. The configuration of the patient's mask and endotracheal tube are not described here as these are standard across the industry and can be connected to the rest of the gas pathway system 200.

### Gas Intake Control and Mixing Module

The gas intake control and mixing module 210 includes an air intake control assembly 220 and an oxygen intake control assembly 240. The air intake control assembly 220 and the oxygen intake control assembly 240 are adapted for connecting to standard medical air 202 and oxygen 204 supplies, such as station outlets found in hospitals, which typically provide between about 50 and about 60 psi of head pressure. The air intake control assembly 220 includes a first gas line 222 connected to the medical air supply 202 and the oxygen intake control assembly 240 includes a second gas line 242 connected to the oxygen supply 204. The air intake control assembly 220 has a first pressure sensor 224 in-line with the medical air supply 202 for sensing pressure in the first gas line 242 while the oxygen intake control assembly 240 has a second pressure sensor 244 in-line with the oxygen supply 204 for sensing pressure in the second gas line. The pressure sensors 224 and 244 send pressure data for air and oxygen lines to the ventilator control system (as shown in FIG. 1) to determine whether adequate air and oxygen are available. Next, each of the air intake control assembly 220 and the oxygen intake control assembly 240 connects to a respective filter (232 and 252) and a respective control valve (226 and 246). According to some embodiments, the control valves 226 and 246 each has a variable aperture for controlling the flow of air and oxygen, respectively. According to some embodiments, the control valves 226 and 246 are rated for at least 50 psi to accommodate the head pressure. The control valves 226 and 246 enable stepping down the pressure to a level usable by a patient (e.g., under 1 psi pressure). According to some embodiments, the control valves 226 and 246 are controlled by a variable 0-5 VDC analog signal that is proportional to the aperture size of the control valves 226 and 246. The control valves 226 and 246 are controlled via the ventilator control system, which sends a signal across the I2C bus connecting the control unit to the electronics control board. Residing on the electronics control board and connected to the I2C bus, are two digital to analog converters (DACs) that generate precise signal voltages that regulate the control valves. The pressure of the medical air and the oxygen can be controlled by adjusting the aperture size of the control valves 226 and 246, which increases or decreases the pressure and flow rate of gas in the system 200. According to some embodiments, bleed valves 228 and 248 are added after the control valves 226 and 246, respectively, to allow venting excess pressure within the respective intake control assemblies 220 and 240. Each of the variable valves 226 and 246 has a corresponding bleed valve (228 and 248), enabling the pressure for medical air and oxygen to be controlled independently. Toggling the bleed valves 228 and 248 provides an additional mechanism for adjusting the pressure in the gas intake control and mixing module 210, which allows adjusting the system pressure to the atmospheric level. After the bleed valves 228 and 248, air intake control assembly 220 and oxygen intake control assembly 240 are each fitted with a flow meter (230 and 250) to measure the flow rate of medical air and oxygen, respectively. According to some embodiments, the flow meters 230 and 250 each produce a 0-5 VDC signal, when they are operating in analog mode, that is sampled by an analog to digital to converter (ADC) chip 124 (as shown in FIG. 1). The digital output of the ADC chip 124 can then be read digitally by the I2C bus 114 (as shown in FIG. 1). According to some other embodiments, when the flow meters are operating in digital mode, the flow meters 230 and 250 can produce digital readings outputted to the I2C bus directly. According to some additional embodiments, the flow meters 230 and 250 do not measure volume directly, instead they measure mass flow in L/min. The gas intake control and mixing module 210 joins and/or mixes the medical air and oxygen from the gas lines 222 and 242 to produce oxygenated air, which is then delivered through the patient delivery module 260 to the patient. As will be described in detail below, the patient delivery module 260 senses the pressure of the oxygenated air delivered to the patient and outputs the pressure data to the control system (see FIG. 1). Based on the pressure data outputted to the control system, the pressure in the gas lines 222 and 242 can be controlled by adjusting the variable control valves (226 and 246) and the bleed valves (228 and 248) jointly. Additionally, the flow rate of each gas line 222 and 242 can be controlled separately by adjusting the respective variable valve (224 and 244) and the respective bleed valve (228 and 248). The relative flow data between medical air and oxygen can be used to determine the proportion of oxygen in the gas mix that is delivered to the patient. The flow of each gas line can be finely adjusted to keep the oxygen proportion in the oxygenated air at a set value. For example, micro-adjustments can be made to the various components at 50 millisecond or smaller increments, as needed.

### Patient Delivery Module

According to some embodiments, the patient delivery module 260 includes a patient valve 266 with controllable aperture (e.g., a solenoid valve), a system side pressure sensor micro-nozzle 264, a patient-side pressure sensor micro-nozzle 268, and a protective filter 270 (e.g., a HEPA filter) placed between gas pathway system 200 and the patient. The patient valve 266 functions as a gatekeeper for all mixed gas (i.e., oxygenated air) that goes into the patient. The patient valve 266 is closed by default and opens to let oxygenated air into the patient only when the timing and certain conditions are met. If the conditions are incorrect or if the variable valves 226 and 246 have leakage or are defective, the patient valve 266 will prevent inadvertent patient ventilation. According to some examples, a pressure sensor micro-nozzle 264 is placed along gas line 261 between the gas intake control and mixing module 210 and the patient valve 266 to sample gas pressure in this part of the system. According to some embodiments, the pressure sensor micro-nozzle 264 connects, via tubing, to a digital I2C pressure sensor with a corresponding micro-nozzle residing on the electronics control board, thus allowing direct sampling of the air pressure in gas line 261. According to additional embodiments, a pressure sensor micro-nozzle 268 is placed along gas line 262 between the patient valve 266 and the protective filter 270 to sample gas pressure in the patient's lungs. According to some embodiments, the pressure sensor micro-nozzle 268 connects, via tubing, to a digital I2C pressure sensor with a corresponding micro-nozzle residing on the electronics control board, thus allowing direct sampling of the air pressure in gas line 262.

### Exhalation Module

According to some embodiments, the exhalation module 280 includes a protective filter 281 (e.g., a HEPA filter), a gas line 282, a pressure sensor micro-nozzle 284, a flow meter 286, a gas line 287, a PEEP assembly 288, and a CO2 sensor 289. The gas line 282 has a first end adapted to receive exhaled air from the patient via the protective filter 281 and a second end adapted to expel the exhaled air via the PEEP assembly 288. The PEEP assembly 288 in turn expels a small portion of the exhaled air via the CO2 sensor 289 to sample the exhaled air for CO2 content.

According to some embodiments, the pressure sensor micro-nozzle 284 is placed along the gas line 282. The micro-nozzle 284 is connected, via tubing, to a digital pressure sensor with a corresponding micro-nozzle residing on the electronics control board, thus allowing direct sampling of the air pressure in the gas line 282. The air pressure in the gas line 282 is indicative of the air pressure in the patient's lungs during exhalation. According to some embodiments, the flow meter 286 is configured for detecting the flow rate of the exhaled air through the gas line 282. For example, when operating in analog mode, the flow meter 286 can output flow data for the exhaled air via an analog 0.5-4.5 VDC signal to an analog-to-digital converter (ADC) 124, which outputs the converted flow data via the I2C bus 114 (as shown in FIG. 1). Alternatively, when operating in the digital mode, the flow meter 286 can be configured to produce digital readings outputted to the I2C bus 114 directly, when the flow meter 286 is operating in digital mode.

According to some embodiments, the PEEP assembly 288 further includes a manifold 298 that divides the gas line 287 into two branching sections-a first branch 296 is connected to a rapid valve 290 (i.e., a first valve) while a second branch 294 is connected to a slow valve 292 (i.e., a second valve). According to some embodiments, the PEEP assembly 288 includes a rapid valve 290 with a high flow rate and a slow valve 292 with a lower flow rate relative to the high flow rate. The rapid valve 290 is configured to release most of the air from of the patient's lungs at a fast rate, while the slow valve 292 slowly releases the remaining air in the patient's lungs at a slower rate to maintain a positive end-expiratory pressure in the lungs of the patient once the rapid valve 290 is closed. According to some embodiments, the rapid valve 290 and the slow valve 292 are solenoid valves, the opening and closing of which can be controlled by the control unit 110 via the input/output (I/O) pins 116 and the switch controller 128 (as shown in FIG. 1). According to some embodiments, the slow valve 292 has a smaller effective aperture than the rapid valve 290. For example, the rapid valve 290 can be configured to open its apertures fully or near-fully to release exhaled air out of the patient quickly. In comparison, according to some embodiments, the slow valve 292 can include a flow limiter to reduce its effective aperture and thus the flow rate. Alternatively, or additionally, flow rate through the slow valve 292 can restricted by configuring the second branch 294 to have a narrower diameter than the first branch 296. Other means of restricting the flow rate of the slow valve 292 relative to the rapid valve 290 would be readily apparent to a person of ordinary skill in the art. Operation of the PEEP exhale valve assembly 288 during an exhale cycle is described in detail in the sections below.

According to some embodiments, the exhalation module 280 further includes a carbon dioxide (CO2) sensor 289 configured to detect the CO2 level in the exhaled air. For example, the CO2 sensor 289 can be connected to a release terminal of the slow valve 292. The CO2 sensor 289 is configured to analyze the exhaled air and output a CO2 level detected in the exhaled air to the control unit 110 via the I2C bus 114 (as shown in FIG. 1).

### 3D Printing and Injection Molding of Components

The present disclosure also discloses the use of 3D printing or injection molding to manufacture some or all the components for the gas delivery system. For example, all of the connecting elements between the various components (e.g., the one or more micro-nozzles connecting to the pressure sensor chips) can be miniaturized using 3D printing or injection molding to eliminate gas fittings and make connections between components more direct and compact. Using 3D printing could also reduce the cost of production and turn-around time for procuring replacement parts for the ventilator system. The way in which these elements are designed are unique to the disclosed ventilator system.

### Ventilator Operation Cycle

The present disclosure also discloses the operation cycle of the above-described ventilator system, which divides each ventilation cycle into three sub-cycles—the inhale cycle, the exhale cycle, and the waiting cycle.

### Inhale Cycle

During the inhale cycle, the energy required to push the mixture of air and oxygen (collectively referred to as “oxygenated gas”) into the patient is derived from the compressed nature of the gas supplied to the gas pathway system 200. As previously described, the gas intake control and mixing module 210 relies on the variable valves 226 and 246 to control: a) the flow rate at which oxygenated gas passes into the patient, b) the pressure of the oxygenated gas, c) the volume of the oxygenated gas, and d) the mixing ratio of oxygen to medical air for the oxygenated gas. Feedback data from a series of sensors placed downstream in the gas pathway system 200 enable the variable valves 226 and 246 to finely control ventilator flow parameters. For example, such sensors can include the flow meters 230, 250, and 286, and corresponding pressure sensors connected to the micro-nozzles 264, 268, and 284. The collection of sensors output continuous data to the control unit 110 (as shown in FIG. 1) to enable dynamic control of the aperture opening of the variable valves 226 and 246. The system determines the inhale cycle is complete when the patient's lungs achieve certain preset volume or pressure parameters setup by the user. Once the inhale cycle is complete, valves 226, 246, and 266 are closed to shut off gas flow to the patient.

### Exhale Cycle

The exhale cycle begins after completion of the inhale cycle. During the exhale cycle, air in the patient's lungs is released through the exhalation module 280. As previously described, according to some embodiments, the exhalation module 280 includes a protective filter 281, a gas line 282, a pressure sensor micro-nozzle 284, a flow meter 286, a gas line 287, a PEEP assembly 288, and a CO2 sensor 289. Initially in the exhale cycle, both the rapid valve 290 and the slow valve 292 are opened to allow air from the patient's lungs to be quickly exhaled through the opened valves. Once most of the air has been exhaled, the control unit closes the rapid valve 290 while the slow valve 292 remains open to release the remaining air more slowly so as to maintain the end-expiratory pressure in the patient's lungs. According to some embodiments, once the end-expiratory pressure in the lungs has drifted down to a preset value, the control unit closes the slow valve 292, thus completing the exhale cycle. According to some embodiments, the thresholds for when to turn off the rapid valve 290 and the slow valve 292 are predetermined values based on pressure and/or flow rate in the exhalation module 280. For example, according to some embodiments, the control unit 110 (as shown in FIG. 1) of the ventilator system tracks the volumes of air inhaled and exhaled by the patient during a ventilation cycle. For example, to track exhaled air volume, the control unit 110 uses multiple small increments of air flow data, supplied by the flow meter 286, taken successively over small increments of time (50 ms or smaller), spanning the exhale cycle. This process generates a series of small volumes, added together in real time, which enables the control unit 110 to know the exact current volume of exhaled air during each cycle. The control unit 110 then compares the volume of exhaled air to the volume of air previously inhaled into the patient's lungs. According to some embodiments, the volume of air previously inhaled into the patient's lungs can be determined based on flow data taken from the flow meters 230, 250 and time increment measurements similar to the method described above. When the volume of exhaled air reaches a first predetermined proportion of the volume of the previously inhaled air (e.g., defined as a percentage of the inhaled air), the control unit 110 triggers the closure of the rapid valve 290.

After the rapid valve 290 is closed, air continues to be exhaled from the patient's lungs through the slow valve 292. Closure of the rapid valve 290 significantly slows the flow rate of the air being exhaled from the patient's lungs, and as a result, significantly slows the rate of the pressure drop occurring in the exhale tubing. According to some embodiments, the control unit 110 continuously compares the pressure in the patient's lungs to a predetermined PEEP pressure. According to some embodiments, the pressure in the patient's lungs can be indicated by the pressure in the gas line 282. According to some embodiments, the pressure from the gas line 282 is indicated by the continuous pressure data from a pressure sensor connected to the micronozzle 284. When the pressure in the patient's lungs or the pressure in the gas line 282 drifts to the predetermined PEEP pressure, the control unit 110 triggers the closure of the slow valve 292. By slowing the rate of pressure drop prior to achieving the PEEP pressure, the system can reduce the chances of overshooting or undershooting the final PEEP pressure relative to the user defined PEEP pressure. The exhale cycle is complete when both the rapid valve 290 and the slow valve 292 are shut off.

### Waiting Cycle

After the current exhale cycle ends and before the next inhale cycle begins, or when the ventilator system goes into a standby mode, the ventilator system idles in the wait cycle. During the wait cycle, the control unit 110 monitors sensor data from the collection of sensors until the trigger conditions for starting the next inhale cycle are met. The trigger conditions in the wait cycle are configured to depending on the control mode that the ventilator is operating in. For instance, when the ventilator system is set to the volume control mode, the ventilator is set to wait for a specific time point to trigger an inhale cycle. Alternatively, when the ventilator is operating in the CPAP mode, the control unit is configured to trigger inhalation cycle based on a drop in the airway pressure as detected by pressure sensor 268, which signifies that the patient is attempting to breathe. In yet another alternative, when the ventilator is operating in the SIMV mode, the control unit is configured to trigger the inhale cycle by either a patient-initiated breath or a time point trigger. When the ventilator is in the standby mode it does not respond to any triggers.

### Software Architecture

According to an embodiment, the ventilator software is organized using three overlapping organizational schemas which describe the integrative function of the software. These three schemas are: 1) object classes, 2) multi-threading, and 3) ventilator state data flow. Additionally, the present disclosure describes a smart assistant functionality that will recommend initial settings for the ventilator and make ongoing suggestions/changes to the ventilator settings based on patient conditions, such as the patient's blood gas values. Further, the present disclosure also describes a software watchdog functionality that acts as a failsafe mechanism and detects software crashes

### Object Classes

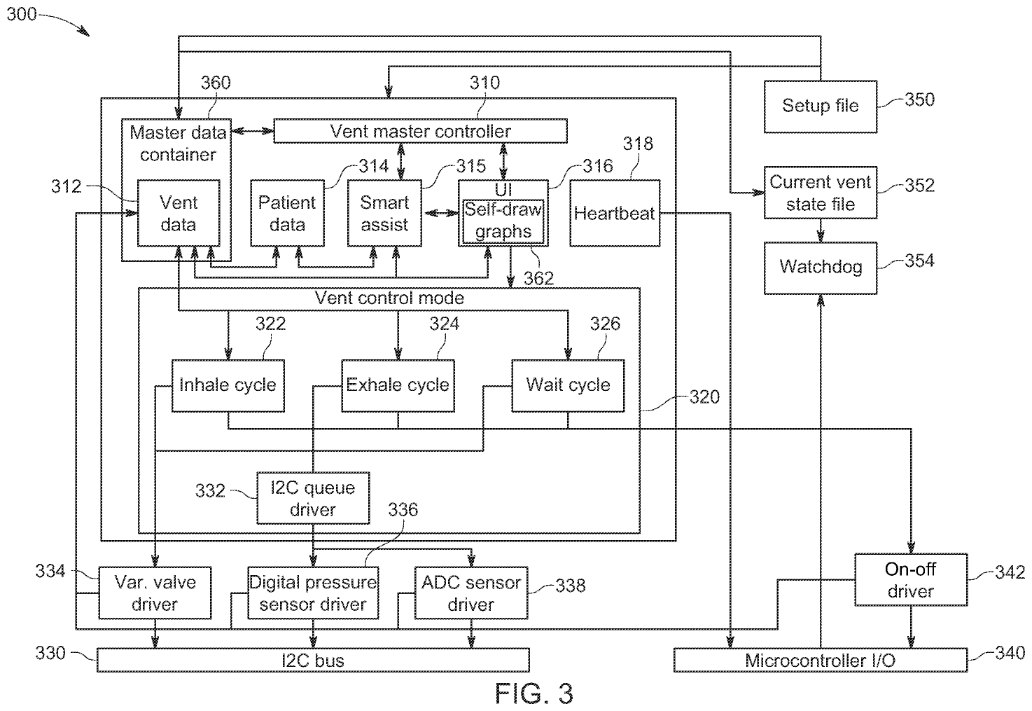

FIG. 3 illustrates an exemplary control circuit for the control software 300 used to control the ventilator system in FIG. 1. The first organizational level is at the class level. According to some embodiments, the master controller class 310 contains and owns everything from the sensor drivers 336 and 338, the valve drivers 334, the queue driver 332 for organization of the I2C bus traffic, the user interface 316, the smart assistant 315, and the ventilator control mode classes 320, which drive the ventilation.

Upon startup, the ventilator detects which hardware has been installed and selects the appropriate driver to run the detected hardware. The hardware can include any sensors or valves that the ventilator has been installed with. Once the master controller receives a ventilation control mode selection, the master controller 310 creates an instance of that ventilation control mode 320. The new instance of the selected ventilation control mode then creates new instances of the appropriate inhale 322, exhale 324, and wait cycles 326 which are context-specific to the particular ventilation control mode 320. The new instance of the ventilation control mode 320 instructs each of the objects (e.g., objects for inhale cycle 322, exhale cycle 324, and wait cycle 326) to perform their respective functions in a loop until the master controller 310 receives user instruction to stop the master controller 310 to instruct the instance of the ventilation control mode 310 to stop.

### Multi-Threading

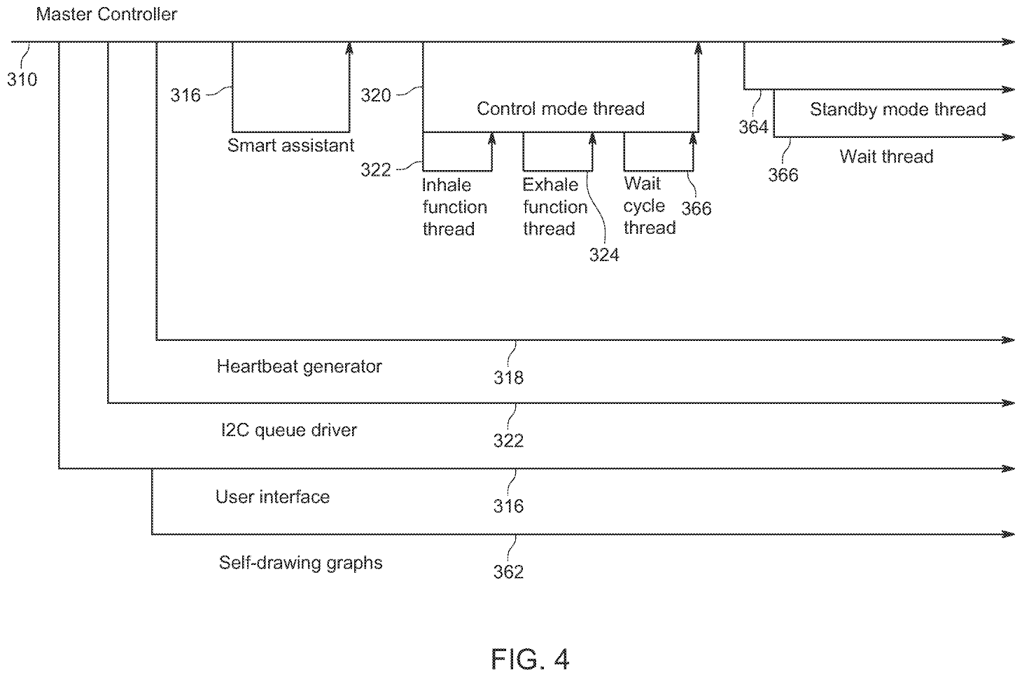

On the second level of organization, the present application discloses a ventilator software with instructions that, when executed, can compartmentalize the collection of functions into multiple computational threads (i.e., interchangeably, also called threads) that can be instantiated simultaneously. Each thread can be thought of as a living instance of multiple classes coming together on an ongoing basis to perform a particular job. FIG. 4 illustrates exemplary threads for a disclosed ventilator system executing in parallel with each other over a period of time. The master controller thread 410 instantiates at the start of the ventilator software. The master controller thread 410 instantiates, manages, and deconstructs a collection of slave threads. For instance, the slave threads cand include one or more of a heartbeat generator 420, an I2C queue driver 430, a graphical user interface 440, a smart assistant 450, and one or more control modes 460 for the ventilator. The heartbeat generator thread 420 periodically or continuously receives status signals from one or more threads that indicate whether one or more of the other threads are running as expected. The heartbeat generator thread 420 then determines whether each of the status signals received from the one or more thread passes a status check. The heartbeat generator thread 420 continuously or periodically generates a signal (i.e., “heartbeat signal”) while the one or more status signals from the one or more threads continue to pass the status checks. The I2C queue driver 430 continuously requests and schedules the order that data is received from the sensors, such as the various flow meters, pressure sensors, and the carbon dioxide monitor. According to some embodiments, the I2C queue driver 430 periodically or continuously queries one or more sensor drivers for sensor data. Each sensor driver, in turn, requests sensor data over the I2C bus from its paired sensor. Each sensor driver then deposits this data into the master data container. According to some embodiments, the master data container holds variables and functions that manage the variables and data structures. The functions of the master data container are triggered by and run by one or more of the threads, such as the independent threads described herein. Therefore, according to some embodiments, the entire process of continuous data collection, including the actions of the sensor drivers and actions or the master data container are triggered by and run inside an instance of the I2C queue driver 430 thread. For a component of the ventilator to retrieve data from the master data container, a computational thread associated with said component runs one or more functions of the master data container to read the requested data directly. The master data container and its functions are described in detail in other parts of the present disclosure.

The user interface (UI) thread 440 includes instructions configured to generate one or more graphical user interfaces on one or more display devices associated with the ventilator. According to some embodiments, the UI thread 440 enables a user to input data via one or more graphical user interfaces into the ventilator system 100 (see FIG. 1) and display one or more types of data on the one or more display devices. For example, the UI thread 440 includes instructions configured to receive user-inputted data (e.g., patient data and setting parameters) via one or more user interfaces and store the user-inputted data to the master data container 360 or a patient data repository 314 (see FIG. 3). According to some embodiments, the master controller thread 410 or the UI thread 440 is configured to further instantiate a self-drawing graphs thread 442 that includes instructions configured to plot or visualize data on the one or more display devices. For example, the master controller thread 410 or the UI thread 440 instantiates the self-drawing graphs thread 442, which retrieves sensor data or data synthesized based on the received sensor data from the master data container, and plots the retrieved data on one or more display devices. The smart assistant thread 450 enables the ventilator to recommend initial settings and/or setting adjustments to the ventilator based on input data, such as received sensor readings and/or data input from a user (e.g., the control mode setting for the ventilator, or patient related information). The control mode thread 460 receives input data (e.g., a control mode setting provided by the user) and instantiates sub-threads to control the inhalation cycle, the exhalation cycle, and wait cycle to meet the operating characteristics for each control mode. More specifically, within the inhalation, exhalation, and wait cycles of each control mode, one or more of the aforementioned ventilator components are configured to operate in particular manners. According to some embodiments, each of the cycles can be controlled by an independent corresponding thread. For instance, an inhale function thread 462 controls the inhalation cycle, an exhale function thread 464 controls the exhalation cycle, and a wait cycle thread 466 controls the wait cycle. According to some embodiments, the inhale function thread 462 controls, among other things, air and oxygen mixture ratio, air delivery volume, duration, and pressure. According to some embodiments, the exhale function thread 464 controls, among other things, a first threshold condition for when to turn off the rapid valve 290 and a second threshold condition for when to turn off the slow valve 292 (shown in FIG. 2). According to some embodiments, the wait cycle thread 466 controls, among other things, conditions that would terminate the wait cycle and trigger the next inhale function thread 462. For example, conditions for terminating the wait cycle include one or more of passing of a predetermined duration, voluntary breathing initiated by the patient (when the ventilator is operating in CPAP mode), or other conditions.

According to some embodiments, the threads are configured to run simultaneously and independently from each other. For example, during a ventilation cycle, the following threads can be executed simultaneously: 1) the UI thread 440 continuously updates graphs, on one or more display devices, indicating air pressure, flow rate, and gas volume based on the sensor data retrieved from the master data container; 2) the I2C queue driver 430 continuously requests data from the one or more sensors and deposits that data into the master data container; 3) the inhale function thread 462 accesses the continuously updated sensor data from the master date container to determine if a predetermined condition (e.g., a target gas volume) has been met; and 4) the heartbeat generator 420 continuously receives signals from one or more monitored threads and indicate to a watchdog module whether the monitored threads monitored continue to function. Over the course of the life of one instance of the ventilator process, multiple threads are instantiated, data is handed off from one thread to one or more other threads, and threads are deconstructed in an orchestrated manner.

### Ventilator State Data Flow

On the third level of organization, the present application discloses ventilator software with instructions that, when executed, causes ventilator data (e.g., sensor data, ventilator internal states, error alerts, calculated values) to be stored in a central data repository, such as the ventilator data repository 312 (as shown in FIG. 3). Upon startup of the ventilator software, the master controller 310 creates and houses the master data container. The master data container houses multiple simple and complex data items that the ventilator system needs to keep track of. According to some embodiments, the master data container contains data including the current flow rate through a given flow meter or historical time points of when the patient took a breath. The data objects are constantly being written to by various other threads and objects. According to some embodiments, the master data container also houses multiple functions necessary for the maintenance of the data objects. Such functions include a function that calculates and updates the current total inhaled lung volume based on the current data collected by the oxygen flow meter 250 and the air flow meter 230 (shown in FIG. 2). According to some embodiments, the master data container does not run its own computational thread, but is instead “inert” as its functions and processes are being driven by other threads interacting with it. According to some embodiments, upon startup of the ventilator software, the master controller 310 creates and stores files for one or more hardware drivers, (e.g., one or more sensor drivers) and the I2C queue driver 332. Upon creation of the I2C queue driver 332, the master controller 310 passes a list of all active I2C sensor drivers to the I2C queue driver 332. The master controller 310 then starts the data acquisition process by directing the I2C queue driver 332 to start an independent computation thread that continuously loops and collects data. For example, the I2C queue driver 332 instructs one or more of the active sensor drivers to sample current data from their respective sensors. The I2C queue driver 332 then schedules the order of receiving and receives the sensor data. The I2C queue driver 332 then instructs one or more of the sensor drivers to update the master data container with the most current sensor data. According to some embodiments, the data acquisition process is repeated periodically to ensure that the stored data is current. For example, once the I2C queue driver 332 completes a data acquisition cycle, the I2C queue driver 332 waits for the next time point to query the sensor data and repeats the data acquisition cycle. According to some embodiments, the I2C queue driver 332 is configured to repeat the data acquisition cycle every fifty milliseconds.

According to some embodiments, some forms of data are written to the master data container via consume functions that cause any relevant calculations or generation of any secondary data pieces to occur. Therefore, when data, such as a flow measurement, is stored to the ventilator data repository 312, the function within the ventilator data repository that is tasked with consuming this data calculates the duration since the last measurement and uses the duration value and the flow measurement to determine an incremental volume, that is the volume of gas that passed by the flow meter in that duration. The incremental volume in that duration is added to a total volume list for the flow meter.

According to some embodiments, for the inhale function thread 462 (shown in FIG. 4) to control gas delivery to the patient, the inhale function thread 462 receives the slope values of the pressure and the flow measurements in addition to the actual values in order to make rapid enough decisions to prevent the pressure and the flow from overshooting a target pressure or a target flow. Specifically, the inhale function thread 462 requires both the actual value and the slope value of a line indicative of a change rate of the pressure and the actual value and the slope of a line indicative of a change rate of the flow. As sensor data from a flow meter and a corresponding pressure sensor are deposited into the repository, a calculation is made regarding the slope of the increase or decrease. The slope is then added to and tracked in an array stored in the ventilator data repository 312 (shown in FIG. 3). The ventilator data repository 312 is responsible for constantly tracking multiple pieces of data and calculations. At the end of the data journey, the data is accessed by one or more elements of the ventilator software to understand the state of the ventilator system. In this embodiment, an independent computational thread that controls pushing gas into the patient can further perform data safety check before pushing gas into the patient. For example, before allowing enlarging the variable valve aperture 266 (as shown in FIG. 2), the independent computational thread can check one or more parameters (e.g., slopes of pressure and flow), by directly reading stored data from the master data container 360, to ensure that the values of the one or more parameters are over their predefined specifications. According to some embodiments, this independent computational thread also uses ventilator volume data to stop the ventilator cycle. According to some embodiments, the user interface thread 442 and the self-drawing graphs thread 442 access the data stored in the master data container to make continuous graph tracings and to populate the numbers on the buttons that reflect the current settings. According to some embodiments, each of the independent threads described herein read data variables directly from the master data container or read data variables through functions housed within the master data container designed to aid in consumption of those variables.

### Smart Assistant Functionality

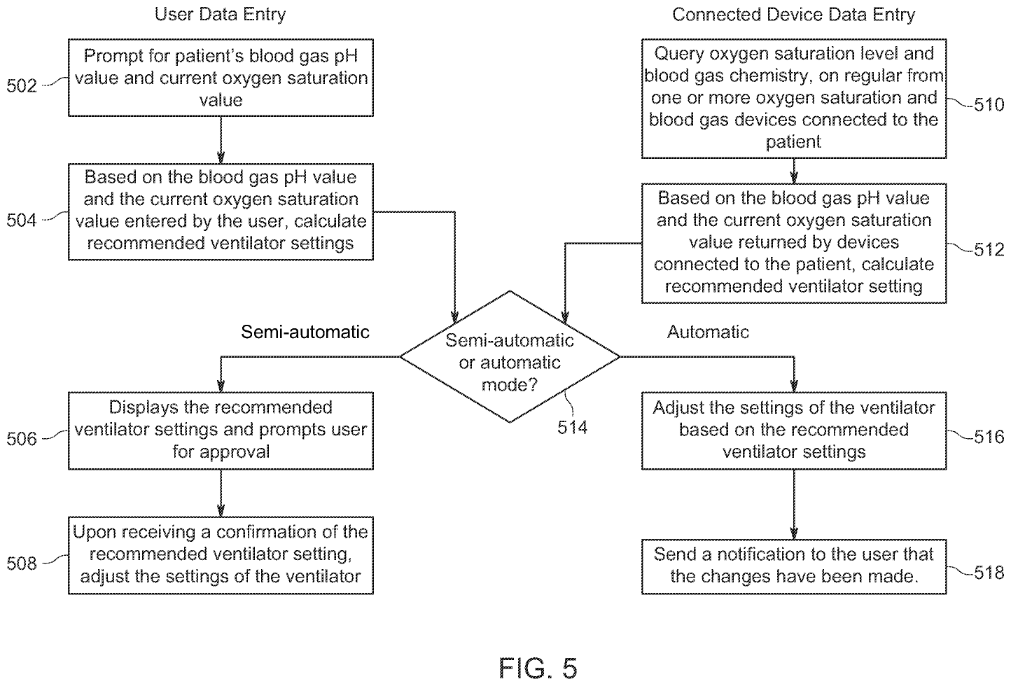

The present disclosure further discloses a “smart assistant” functionality that allows the ventilator to recommend ventilator settings or automatically adjust settings on the ventilator based on measured data and qualitative data provided to the ventilator control system. Conventionally, ventilator settings are initially determined by an ICU or anesthesia physician. Based on the pH value, the dissolved CO2 and O2 content derived from the patient's blood gas analysis, ongoing adjustments are made to one or more ventilator settings, including the ventilator oxygen mix (i.e., FiO2), PEEP setting, delivered volume setting (i.e., tidal volume), respiratory rate, and the amount of pressure support to the patient. This analysis is critical to ongoing ventilator support. However, making timely determinations of ventilator settings and adjusting as the patient's lung conditions change could become difficult to achieve if physicians are unavailable. The smart assistant is an automated setting-suggestion functionality that recommends initial settings for the ventilator and makes ongoing suggestions and/or changes to the ventilator settings based on the blood gas values of the patient. FIG. 5 illustrates an exemplary block diagram of a process for the ventilator system to suggest and set or adjust ventilator settings. According to some embodiments, the ventilator system “smart assistant” feature sets initial ventilator settings. According to some embodiments, the ventilator smart assistant can either acquire the blood gas data from user entry or from one or more devices connected to the patient. In the embodiment where the ventilator is acquiring blood gas data from user entry, the algorithm begins at step 502. At step 502, the ventilator system prompts, via a user interface, for input of the patient's blood gas pH value and current oxygen saturation value. At step 503, based on the blood gas pH value and the current oxygen saturation value entered by the user, the ventilator system calculates recommended ventilator settings. At step 512, the smart assistant can be setup to operate in semi-automatic or automatic mode. In the embodiment where the smart assistant is operating in semi-automatic mode, at step 504, the ventilator system displays the recommended ventilator settings via the user interface and prompts the user for approval of the recommended ventilator settings. At step 506, upon receiving a confirmation of the recommended ventilator setting, the control unit adjusts the settings of the ventilator. In the embodiment where the ventilator is acquiring blood gas data from one or more connected devices, the algorithm begins at step 508. At step 508, the ventilator system, query oxygen saturation level and blood gas chemistry from one or more devices connected to the patient. For example, the one or more devices can include one or more oxygen saturation and blood gas devices. At step 510, based on the blood gas pH value and the current oxygen saturation value returned by the one or more devices connected to the patient, the ventilator system calculates recommended ventilator settings.

According to some other embodiments, the ventilator system smart assistant can be configured to operate in semiautomatic mode or automatic mode as it relates to the implementation of recommended changes to ventilator settings. When the ventilator system is configured to operate in the automatic mode, the ventilator system would typically acquire patient data via the connected device pathway 508. The ventilator smart assistant would then automatically calculate recommended ventilator settings 510, make the adjustments to the ventilator settings 514, and notify the user that the appropriate changes were made 516. As a contrast to the semi-automatic mode, the function in the automatic mode does not require user interaction or approval to make the recommended changes to ventilator settings.

According to some embodiments, when the ventilator system is configured to operate in the automatic mode, the ventilator system automatically acquires patient data, calculates recommended ventilator settings, and automatically adjust the ventilator settings based on the recommended ventilator settings. The ventilator system uses the aforementioned steps 508 and 510 to query oxygen saturation level and blood gas chemistry from one or more devices connected to the patient and calculate recommended ventilator settings based thereon. At step 512, the ventilator system adjusts the ventilator settings based on the recommended ventilator settings. At step 514, the ventilator system sends a notification to indicate the adjusted ventilator settings.

### Software Watchdog

The present application further discloses a software watchdog functionality which 1) monitors the ventilator system for software failure and 2) resets the ventilator system in the event of a software failure. The disclosed ventilator system relies on the proper operation of the software to detect the real-time states of various ventilator components and using the real-time states to continuously adjust ventilator components such as the aperture of valves to control the delivery of oxygen and medical air to the patient. As such, any software failure in the ventilator could expose the patient to life-threatening risks. The disclosed ventilator system includes multiple failsafe mechanisms to mitigate the risk of failure, including the software watchdog functionality herein described.

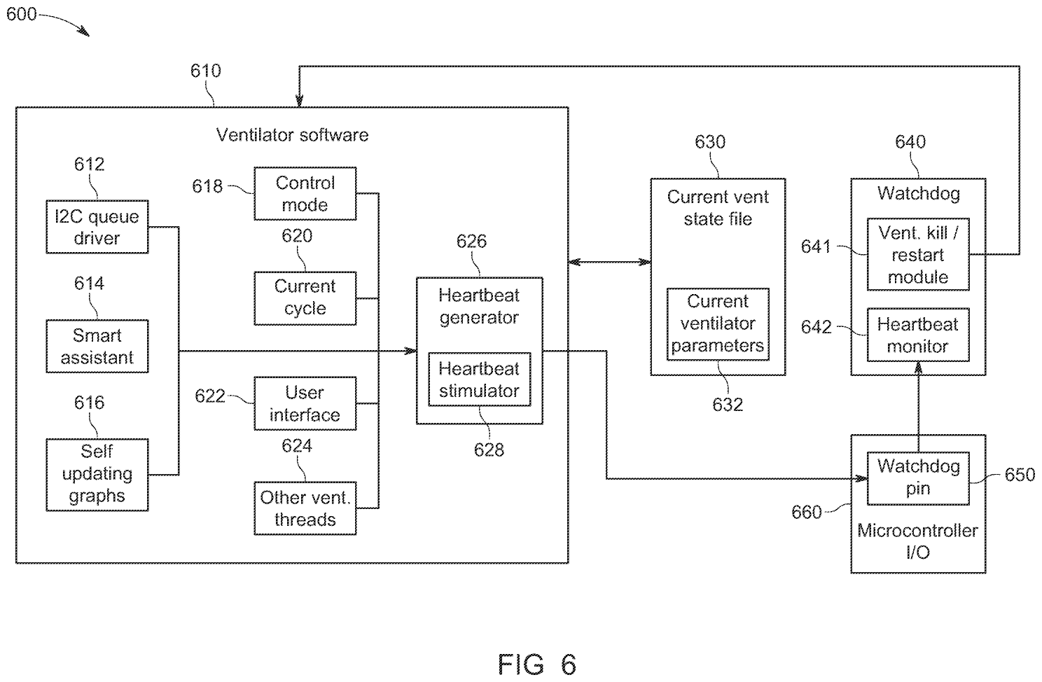

FIG. 6 illustrates a block diagram of an exemplary software watchdog for a ventilator system 600. According to some embodiments, the ventilator software 610 has one or more threads registered to a heartbeat generator class or thread 626. The heartbeat generator 626 generates a continuous or periodic heartbeat signal at a predefined time interval to indicate to a heartbeat monitor thread 642 that the heartbeat generator 626 and the registered threads are functioning. According to some embodiments, the heartbeat generator 626 can be configured to generate a heartbeat signal every 0.5 seconds or less. According to some embodiments, the heartbeat signal can be a signal configured to periodically toggle an I/O pin 650 (i.e., the “watchdog pin”) on a microcontroller 660.

For illustrative purpose, some threads that can be registered to the heartbeat generator 626 include an I2C queue driver 612, a smart assistant 614, a master data container 616, a control mode thread 618, a user interface thread 620. It should be noted that, the group of threads that can be registered to the heartbeat generator 626 is not fixed. Additional threads or a different group than those shown in FIG. 6 can become registered with the heartbeat generator 626. Further, one or more registered threads can also unregister and cease to be monitored by the heartbeat generator 626. For example, a computational thread for a current ventilation cycle 620 (i.e., inhale cycle, exhale cycle, or wait cycle) registers to the heartbeat generator 626 when the thread is instantiated, then becomes unregistered when the thread is de-constructed.

The heartbeat generator 626 continues to generate a heartbeat signal if the heartbeat generator 626 continues to receive check-in signals from all of the registered threads. According to some embodiments, the check-in signal is generated using a heartbeat stimulator function 628. According to some embodiments, the heartbeat generator 626 includes a heartbeat stimulator function 632. Each registered thread can call the heartbeat stimulator function 632 within the registered thread's executable loop. If a registered thread stops calling that function while the thread is still registered to the heartbeat generator 626, then the heartbeat generator 626 will stop generating the heartbeat signal, which in turn triggers a kill/restart module 641, located in the watchdog software 640, to reset the ventilator software 610.

According to an embodiment, a watchdog software 640 has a heartbeat monitor thread 642 configured to monitor whether the I/O pin 650 is being toggled by the heartbeat signal. If the heartbeat signal fails to toggle the I/O pin within a predefined time interval, the heartbeat monitor thread 642 will signal the kill/restart module 641 to reset the ventilator software. As previously discussed, some implementations of the software watchdog use 0.5 seconds as a predefined duration between heartbeat signals. According to some embodiments, the kill/restart module 641 is configured to restart the ventilator software 610 by putting the ventilator into an exhale cycle, closing all valves, and forcibly deconstructing and re-instantiating the ventilator main thread. According to some embodiments, the watchdog software 640 is a separate piece of software that always runs in parallel with the ventilator software 610 to ensure that it is functioning. According to some embodiments, when the ventilator restarts from a forced shutdown, the ventilator system immediately and automatically implements the last stored settings prior to the ventilator's forced shutdown. This can be accomplished by having the ventilator software 610 retrieve the last stored ventilator parameters 632, which, according to some embodiments is stored in a so-called current vent state setup file 630. As an exemplary implementation of the software watchdog has demonstrated, the kill/restart process takes less than 10 seconds to recover from a crash and resume ventilation.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

1. A control unit for controlling a ventilator system, the control unit comprising:
a memory containing machine readable medium comprising machine executable code having stored thereon instructions; and
a control system coupled to the memory comprising one or more processors, the control system configured to execute the machine executable code to cause the control system to:
identify one or more hardware components connected to the ventilator system;
select one or more corresponding drivers to run the one or more identified hardware components;
receive, via a user interface, a ventilator control mode;
create an instance of the ventilator control mode;
create, based on the ventilator control mode, an instance for each of an inhale cycle, an exhale cycle, and a wait cycle, wherein each instance includes instructions for at least one hardware component of the one or more hardware components; and
signal the instance for each of the inhale cycle, the exhale cycle, and the exhale cycle to perform function in loop.

2. The control unit of claim 1, wherein the control system is further configured to:
receive, via the user interface, a command to stop the ventilator control mode; and
stop the cycles.

3. The control unit of claim 1, wherein the one or more hardware components include at least one of a flow meter, a pressure sensor, a carbon dioxide sensor, and a variable aperture gas valve, and a binary open/close gas valve.

4. The control unit of claim 1, wherein the functions are compartmentalized into a plurality of independent computational threads.

5. The control unit of claim 4, wherein the plurality of independent computation threads include at least one of a ventilator master controller, a ventilator control mode, a scheduler for an inter-integrated circuit bus, an error detection watchdog, a graphical user interface, and a ventilator software signal stimulator.

6. The control unit of claim 5, wherein, for each of the inhale cycle, the exhale cycle, and the wait cycle, a respective independent computational thread of the plurality of independent computational threads is instantiated and de-constructed as needed to complete its specific tasks

7. The control unit of claim 1, wherein the ventilator control mode includes at least one of a volume control mode, a continuous positive airway pressure mode, synchronized intermittent mandatory ventilation mode, and a standby mode.

8. The control unit of claim 1 further comprising a heartbeat generator unit configured to generate a periodic electronic signal based on receiving register signal from one or more independent computational threads.

9. The control unit of claim 8 further comprising a heartbeat monitor unit configured to terminate and restart a main ventilator thread with last registered parameters if the periodic electronic signal is not received within a predefined time interval.

10. A positive end-expiratory pressure (PEEP) exhale assembly comprising:
a tubing having a first end adapted to receive exhaled air from a patient and a second end adapted to expel the exhaled air;
a pressure sensor configured to detect an air pressure within the tubing;
a flow meter configured to detect a flow rate of the exhaled air through the tubing;
a valve assembly encapsulating the second end of the tubing, the valve assembly including:
a first valve when opened releases the exhaled air at a first rate; and
a second valve when opened releases the exhaled air at a second rate, the second rate being lower than the first rate; and
a control system configured to toggle the first valve when a plurality of parameters reach a first trigger threshold and toggle the second valve when the plurality of parameters reach a second trigger threshold, the plurality of parameters being based on the air pressure detected by the pressure sensor and the flow rate detected by the flow meter.

11. The PEEP exhale assembly of claim 10, wherein the valve assembly further includes a manifold having an input terminal connected to the second end of the tubing, a first output terminal connected to the first valve and a second output terminal connected to the second valve.

12. The PEEP exhale assembly of claim 10, wherein the first valve has a first aperture and the second valve has a second aperture, the second aperture being smaller than the first aperture.

13. The PEEP exhale assembly of claim 12, wherein the aperture of the second valve is configured to release the exhaled air at a rate for maintaining a positive end-expiratory pressure in lungs of the patient.

14. The PEEP exhale assembly of claim 10, wherein the first valve and the second valve are solenoid type valves.

15. The PEEP exhale assembly of claim 10 further comprising a carbon dioxide monitor configured to detect a carbon dioxide level in the exhaled air.

16. The PEEP exhale assembly of claim 10, wherein the first valve and the second valve are open at initiation of exhalation by the patient.

17. The PEEP exhale assembly of claim 10, wherein the first valve closes when, based on measurement taken by the flow meter, a volume of exhaled air reaches a predetermined proportion of a volume of previously inhaled air.

18. The PEEP exhale assembly of claim 10, wherein the second valve closes when a pressure in the tubing decrease to a predetermined positive end-expiratory pressure.

19. The PEEP exhale assembly of claim 18, wherein the pressure in lungs of the patient is indicated by pressure data from the pressure sensor.

20. A method of controlling exhalation of a patient by mechanical ventilator, the method comprising:
providing a positive end-expiratory pressure (PEEP) valve assembly including:
a tubing having a first end adapted to receive exhaled air from a patient and a second end adapted to expel the exhaled air;
a pressure sensor configured to detect an air pressure within the tubing;
a flow meter configured to detect a flow rate of the exhaled air through the tubing; and
a valve assembly encapsulating the second end of the tubing, the valve assembly including:
a first valve when opened releases the exhaled air at a first rate; and
a second valve when opened releases the exhaled air at a second rate, the second rate being lower than the first rate;
detecting, via the pressure sensor, the air pressure within the tubing;
detecting, via the flow meter, the flow rate of the exhaled air through the tubing;
opening, via a control system, the first valve and the second valve when a trigger event is registered by the control system;
closing, via a control system, the first valve when a volume of exhaled air reaches a predetermined proportion of a volume of previously inhaled air, wherein the volume of exhaled air is based on the flow rate detected by the flow meter; and
closing, via the control system, the second valve when the pressure in the tubing, as measure by the pressure sensor, reaches a predetermined PEEP pressure.
